SEQUENCE LISTING (3 sequences)

SEQ ID NO: 1 - protein, 290 aa, Homo sapiens
MGSSHHHHHHSSGLVPRGSHMNEEFRPEMLQGKKVIVTGASKGIGREMAYHLAKMGAHVVVTARSKETLQKVVSHCLELGAASAHYIAGTMEDMTFAEQFVAQAGKLMGGLDMLILNHITNTSLNLFHDDIHHVRKSMEVNFLSYVVLTVAALPMLKQSNGSIVVVSSLAGKVAYPMVAAYSASKFALDGFFSSIRKEYSVSRVNVSITLCVLGLIDTETAMKAVSGIVHMQAAPKEECALEIIKGGALRQEEVYYDSSLWTTLLIRNPCRKILEFLYSTSYNMDRFINK

SEQ ID NO: 2 - protein, 289 aa, Rattus norvegicus
MGSSHHHHHHSSGLVPRGSHMNEEFRPEMLQGKKVIVTGASKGIGREMAYHLSKMGAHVVLTARSEEGLQKVVSRCLELGAASAHYIAGTMEDMAFAERFVVEAGKLLGGLDMLILNHITQTTMSLFHDDIHSVRRSMEVNFLSYVVLSTAALPMLKQSNGSIAIISSMAGKMTQPLIASYSASKFALDGFFSTIRKEHLMTKVNVSITLCVLGFIDTETALKETSGIILSQAAPKEECALEIIKGTVLRKDEVYYDKSSWTPLLLGNPGRRIMEFLSLRSYNRDLFVS

SEQ ID NO: 3 - protein, 298 aa, Cavia porcellus
MGSSHHHHHHSSGLVPRGSHMNEKFRPEMLQGKKVIVTGASKGIGREIAYHLAKMGAHVVVTARSKEALQKVVARCLELGAASAHYIAGSMEDMTFAEEFVAEAGNLMGGLDMLILNHVLYNRLTFFHGEIDNVRKSMEVNFHSFVVLSVAAMPMLMQSQGSIAVVSSVAGKITYPLIAPYSASKFALDGFFSTLRSEFLVNKVNVSITLCILGLIDTETAIKATSGIYLGPASPKEECALEIIKGTALRQDEMYYVGSRWVPYLLGNPGRKIMEFLSAAEYNWDNVLSNEKLYGRWA

(19) United States
(12) Patent Application Publication
(10) Pub. No.: US 2005/0186659 A1
(43) Pub. Date: Aug. 25, 2005

(54) Methods for protein purification

(76) Inventors: Lars Abrahmsen, Bromma (SE); Joakim Nilsson, Danderyd (SE); Udo Oppermann, Enskede (SE); Stefan Svensson, Stockholm (SE)

Correspondence Address: FISH & RICHARDSON PC, 225 FRANKLIN ST, BOSTON, MA, 02110, US

(21) Appl. No.: 11/040,244

(22) Filed: Jan. 20, 2005

Related U.S. Application Data
(60) Provisional application No. 60/576,777, filed on Jun. 2, 2004.

(30) Foreign Application Priority Data
Jan. 20, 2004 (SE) 0400113-7
Mar. 3, 2004 (SE) 0400528-6

(51) Int. Cl. C12N 9/48; C07H 21/04; C12N 1/21; C12N 15/74

(52) U.S. Cl. 435/69.1; 435/252.33; 435/488; 435/212; 536/23.2

(57) ABSTRACT

The present invention relates to process for the production of a soluble polypeptide having at least one ligand binding site, the process comprising (i) providing a host cell comprising a nucleic acid sequence encoding the soluble polypeptide; (ii) culturing the host cell under conditions whereby the polypeptide is produced, wherein the cell culture medium comprises a non-proteinaceous ligand capable of binding to a ligand binding site of the polypeptide; and (iii) recovering said polypeptide.

### CROSS REFERENCE TO RELATED APPLICATIONS

This application claims priority from Swedish Patent Application No. 0400113-7, filed Jan. 20, 2004, Swedish Patent Application No. 0400528-6, filed Mar. 3, 2004, and U.S. Provisional Patent Application No. 60/576,777, filed Jun. 2, 2004. The prior applications are incorporated herein by reference in their entirety.

### TECHNICAL FIELD

The present invention relates to processes for the production and purification of soluble recombinant polypeptides, such as soluble 11β-hydroxysteroid dehydrogenase type 1 (11β-HSD1).

### BACKGROUND

The main active glucocorticoid hormone in humans is cortisol, and in rodents corticosterone. Over 90% of the active glucocorticoid is bound in circulation, mainly to the corticosteroid binding globulin, whereas the inert counterpart cortisone (11-dehydrocorticosterone in rodents) is unbound (1). The enzyme 11β-hydroxysteroid dehydrogenase type 1 (11β-HSD1) increases intracellular glucocorticoid hormone levels by converting cortisone (11-dehydrocorticosterone) into cortisol (corticosterone) through its 11β-oxidoreductase activity. This pre-receptor activation of glucocorticoids is in analogy with other steroid hormones where two or more enzymes act as metabolic “switches” between inactive and active receptor-binding forms of a hormone (2-6). The glucocorticoid “shuttle” mechanism consists of 11β-hydroxysteroid dehydrogenases mediating the reversible oxo-reduction/hydroxy-dehydrogenation at position C11 of cortisone and cortisol. The two known 11β-HSD isozymes, type 1 and type 2, mediate activation or inactivation of the hormone in a tissue-specific manner, respectively (1, 7). Recently, 11β-HSD1 has gained attention as a possible drug target for reducing the tissue-specific effects of glucocorticoids associated with diabetes and obesity (8, 9).

11β-HSD1 belongs to the family of short-chain dehydrogenase/reductase (SDR) gene superfamily of proteins (10). The structures of several related enzymes (with 15-20% sequence identities) have been determined including human estradiol 17β-dehydrogenase type 1 (17β-HSD1) (11), Streptomyces hydrogenans 3α, 20β-hydroxysteroid dehydrogenase (12), E. coli 7α-hydroxysteroid dehydrogenase (13) and dihydropteridine reductase (14). Each of these enzymes is either a dimer or tetramer, as most other characterized SDR enzymes. None of these enzymes is membrane bound or glycosylated, whereas 11β-HSD1 is a 34 kDa glycosylated membrane protein, attached to the endoplasmic reticular membrane and facing the luminal compartment. As expected, the isolation of full-length 11β-HSD1 to high purity with retained enzymatic activity is challenging (15, 16). Attempts to purify full-length variants of 11β-HSD1 have relied on exploitation of solubilization by detergents to extract the protein from the ER membrane and to prevent nonspecific aggregation during purification (15, 19, 32, 33).

11β-HSD1 is bidirectional in vitro, but is believed to predominantly function as an oxidoreductase in vivo, at least in fully differentiated cells (17, 18). Surprisingly, the oxidoreductase activity of 11β-HSD1 is more sensitive to inactivation than is the dehydrogenase activity, both in cell extracts and following purification of the enzyme (19, 20).

Native 11β-HSD1 is an N-linked glycosylated protein bound to the membrane of the endoplasmic reticulum through a single amino-terminal transmembrane segment. The highly hydrophobic nature of the enzyme has so far prevented its purification to homogeneity in quantities sufficient for detailed structural and functional studies. It has been shown that 11β-HSD1 can be expressed, although in moderate amounts, without the transmembrane segment in an active state in Escherichia coli (21, 22).

Inhibitors (antagonists) of 11β-HSD1 are known in the art from, e.g., WO 01/90090; WO 01/90091; WO 01/90094; WO 01/90092; WO 03/043999; WO 03/044000; and WO 03/044009. Arylsulfonamidothiazoles have been identified as potent and selective inhibitors of 11βHSD1, and as a new class of potential antidiabetic drugs (35).

It is known in the art that enhanced levels of recombinant proteins can be obtained by co-overexpression of the chaperonin GroEL/ES (27-29). The chaperonin is believed to prevent the aggregation of partially folded or misfolded forms of a protein and thereby keep it competent for productive folding.

### BRIEF DESCRIPTION OF THE DRAWINGS

FIGS. 1A and 1B depict expression of soluble 11β-HSD1 as analyzed by SDS-PAGE. Cleared lysates from induced cultures of BL21(DE3) containing pET28a derivatives for expression of human (FIG. 1A) and rat (FIG. 1B) 11β-HSD1. Lane 1, SeeBlue Plus2 pre-stained protein standard. lane 2, overexpression of 11β-HSD1; lane 3, co-overexpression of 11β-HSD1 and GroEL/ES; lane 4, overexpression of 11β-HSD1 in presence of the inhibitor BVT.24829; lane 5, co-overexpression of 11β-HSD1 and GroEL/ES in presence of BVT.24829.

FIG. 2 is an SDS-PAGE analysis of the purification steps for recombinant human 11β-HSD1. Coomassie Blue-stained NuPAGE 10% Bis-Tris polyacrylamide gel (Invitrogen, Carlsbad, Calif.). Lane 1, SeeBlue pre-stained protein standard; lane 2, cleared lysate from an induced culture of BL21(DE3) expressing 11β-HSD1 and GroEL/ES; lane 3, peak fraction eluted from an IMAC column after addition of 0.05% Triton X-100; lane 4, peak fraction from a Superdex200 gel filtration column.

FIG. 3 is a graph depicting active site titration of highly purified, homogenous human 11β-HSD1. Fractional velocities (vᵢ/v₀) of cortisol dehydrogenation by 11β-HSD1 were determined in presence of increasing amounts of the selective and potent inhibitor BVT.24829. Values obtained were fitted to the equation as defined by Morrison, yielding a value for the enzyme active site concentration of 0.36±0.04 μM and a Kᵢ^{app} of 32±1 nM. Total protein concentration was determined by amino acid analysis (triplicate measurements) assuming a molecular mass of 31.9 kDa for the His-tagged enzyme.

FIG. 4 is a graph depicting inactivation of recombinant human 11β-HSD1-catalyzed cortisol dehydrogenation with GuHCl. Protein concentration was approximately 5 μM in 50 mM Tris-HCl, pH 8.0. Solutions were exposed to denaturant, and protected by additives as indicated, for 16-18 h at 4° C. (∘) no additives; (□) 50 μM NADP⁺; (□) BVT.24829 inhibitor and 50 μM NADP⁺.

FIGS. 5A-5E are graphs depicting sedimentation coefficient distribution analysis of recombinant guinea pig 11β-HSD1 at various GuHCl concentrations. The sedimentation velocity data were analyzed as described in “Experimental Procedures”. (5A) no GuHCl; (5B) 0.2 M GuHCl; (5C) 0.4 M GuHCl; (5D) 0.6 M GuHCl; (5E) 0.8 M GuHCl.

### DISCLOSURE OF THE INVENTION

It has surprisingly been found that accumulation of soluble human 11β-HSD1 is enhanced when the culture media during gene expression is supplemented with a potent and selective inhibitor of the enzyme. To the best of the inventors' knowledge, this is the first report of a significant increase in accumulation of soluble protein by addition of a low molecular weight inhibitor during protein synthesis.

According to the present invention, it has also been found that the soluble portion of recombinant 11β-HSD1 expressed in E. coli is found mainly as multimeric aggregates in the absence of a solubilizing system, and to a large extent associated with the endogenous chaperonin GroEL and other E. coli proteins. By co-overexpressing GroEL/ES and adding an 11β-HSD1 inhibitor during protein synthesis, the accumulation of soluble 11β-HSD1 has been increased more than an order of magnitude. Using monodispersity as a screening criterion, the purification process has been improved by evaluating various solubilizing systems for the chromatographic steps, finally obtaining stable monodisperse preparations of both human and guinea pig 11β-HSD1.

By analytical ultracentrifugation, it has been shown that 11β-HSD1 mainly exists as a dimer in the solubilized state. Moreover, by active site titration, using a novel potent inhibitor of the human orthologue, it was possible to monitor the stability of the enzymatic activity and to estimate the fraction of active enzyme molecules in the samples, equaling at least 75% in a typical preparation. Equilibrium unfolding experiments indicate that addition of inhibitor and the cofactor NADPH can stabilize the conformational stability of this enzyme in an additive manner. The present invention paves the way for future X-ray crystallographic studies of 11β-HSD1, and may provide a general method for preparing similar proteins to oligomeric homogeneity with retained biological activity.

Consequently, in a first aspect this invention provides a method for preparing a soluble polypeptide, the method comprising: (i) providing a host cell comprising a nucleic acid sequence encoding a recombinant soluble polypeptide (e.g., a recombinant soluble human polypeptide) comprising at least one ligand binding site; (ii) culturing the host cell under conditions whereby the polypeptide is produced, wherein the cell culture medium comprises a non-proteinaceous ligand that binds to the at least one ligand binding site of the polypeptide; and (iii) recovering the polypeptide from the cell culture medium

The term “recombinant soluble polypeptide” is intended to mean a recombinant polypeptide (e.g., a human recombinant polypeptide) which remains in the supernatant fraction when the insoluble cell debris fraction of disrupted cells is pelletted (removed) by centrifugation. Normally, the amount of soluble polypeptide is increased when the portion of the sequence normally acting as a membrane anchoring sequence is deleted.

The term “non-proteinaceous ligand” is intended to mean a small molecular weight ligand to a polypeptide, such as an enzyme inhibitor (antagonist) or a receptor ligand. Preferably, the non-proteinaceous ligand preferentially binds to the active site of the polypeptide, or bind to a site that overlaps with the binding site of the natural ligand.

Preferably, the said non-proteinaceous ligand is binding to the said polypeptide with a Kᵢ value at least in the micromolar range, such as below 100 μM (for instance between 1 nM and 100 μM) more preferably below 10 μM; below 1 μM; or below 100 nM.

The host cell to be used according to the invention can be a prokaryotic cell, a unicellular eukaryotic cell or a cell derived from a multicellular organism. The host cell can thus e.g. be a bacterial cell such as an E. coli cell; a cell from a yeast such as Saccharomyces cerevisiae or Pichia pastoris, or a mammalian cell. The methods employed to effect introduction of the nucleic acid sequence into the host cell are standard methods well known to a person familiar with recombinant DNA methods.

In some embodiments of the methods described herein, the host cell used in the process also comprises an agent that assists protein folding, such as a molecular chaperone, e.g. the chaperonin designated GroEL/ES. The term “GroEL/ES” refers to the E. coli GroEL-GroES chaperonin system comprising GroEL and its cofactor GroES (For reviews, see e.g. Reference Nos. 36 and 37). Other putatively useful molecular chaperones include e.g. those designated DnaK, DnaJ and GrpE. The host cell can optionally contain a recombinant vector encoding the agent that assists protein folding.

In a further aspect of the invention, the polypeptide is a recombinant soluble human 11β-HSD1 polypeptide comprising (or consisting of or consisting essentially of) amino acids 22 to 290 of SEQ ID NO:1 or a fragment thereof exhibiting oxidoreductase activity. Such a polypeptide can optionally include heterologous sequences such as a tag (e.g., a His tag) for purification of the polypeptide. The non-proteinaceous ligand used in such methods can be an inhibitor of an enzymatic activity of 11β-HSD1 (e.g., an inhibitor of oxidoreductase activity of 11β-HSD1 and/or an inhibitor of dehydrogenase activity of 11β-HSD1).

When the polypeptide is a recombinant soluble human 11β-HSD1 polypeptide, the non-proteinaceous ligand can be an arylsulfonoamidothiazole derivative, e.g. a compound selected from those disclosed in WO 01/90090, WO 01/90091, WO 01/90094, WO 01/90092, WO 03/043999, WO 03/044000, or WO 03/044009. Exemplary non-proteinaceous ligands that can be used in such methods include but are not limited to BVT.24829 (3-chloro-2-methyl-N-{5-methyl-4-[2-(3-oxo-4-morpholinyl)ethyl]- 1,3-thiazol-2-yl}benzenesulfonamide), BVT.3498 (3-chloro-2-methyl-N-{4-[2-(3-oxo-4-morpholinyl)ethyl]-1,3-thiazol-2-yl}benzenesulfonamide), BVT.4584 (N,N-diethyl-(3-{[(4-propylphenyl)sulfonyl]amino}thien-2-yl)carboxamide), and BVT.2733 (3-Chloro-2-methyl-N-{4-[2-(4-methyl-1-piperazinyl)-2-oxoethyl]-1,3-thiazol-2-yl}benzenesulfonamide). The skilled person is able to determine which compounds are suitable for use according to the present invention.

In another aspect, the invention provides a method as defined above, said method in addition comprising subjecting the recovered polypeptide to at least one chromatography step in the presence of (i) a non-proteinaceous ligand that binds to the at least one ligand binding site of the polypeptide; and (ii) a solubilizing agent. The chromatography step can comprise affinity chromatography, such as immobilized metal affinity chromatography (IMAC). Other possible chromatography techniques include e.g. gel filtration, ion-exchange chromatography, chromatofocusing, or hydrophobic interaction chromatography (For review, see e.g. Janson, J-C., and Rydén, L. (Eds.), Protein Purification (2nd edition): Principles, High Resolution Methods, and Applications, John Wiley & Sons, Inc., New York, (1997))

The said solubilizing agent is an agent which solubilizes the polypeptide and maintains it in a single (predominant) oligomeric form in solution. Suitable solubilizing agents include Triton® X-100 (i.e. p-(1,1,3,3-tetramethylbutyl)phenol ethoxylate), but other solubilizing agents, such as CHAPS and GuHCl, are also known to the skilled person.

In another aspect, the invention provides a process for obtaining crystals of 11β-HSD1, comprising the steps of: (i) providing a monodisperse preparation of a recombinant soluble human 11β-HSD1 polypeptide, obtained by a method described herein; and (ii) crystallizing the recombinant soluble human 11β-HSD1 polypeptide. The crystallizing step can be carried out according to methods well known in the art, for review, see e.g. Bergfors, T. M. (Eds.), Protein crystallization: Techniques, Strategies, and Tips, International University Line, La Jolla (1999).

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention will now be further illustrated through the description of examples of its practice. The examples are not intended as limiting in any way of the scope of the invention.

### EXAMPLES

Experimental Procedures

Enzyme activity measurements. The dehydrogenase activity of 11β-HSD1 was assayed at 25° C. in 50 mM Tris/Cl, pH 7.4, using 50 μM cortisol and 100 μM NADP⁺, with a K2 spectrofluorimeter (ISS, Champaign, Ill.). The excitation and emission wavelengths were 340 nm and 460 nm, respectively. To ensure unambiguous initial rate measurements, the enzyme addition was adjusted to give a linear fluorescence increase for at least the first 2 min of reaction. Reaction rates were calculated before and after the addition of purified 11β-HSD1 to give enzymatic rates of NADP⁺ reduction. The relationship between the increase of fluorescence and the rate of NADP⁺ reduction was established by generating a calibration curve using freshly prepared solutions of NADPH (0-25 μM). The reductase activity of 11β-HSD1 was assayed by metabolite determination using HPLC. The enzyme was incubated at 37° C. for 10-60 min in 50 mM Tris/Cl, pH 7.4, containing 50 μM cortisone and 100 μM NADPH or an NADPH-regenerating system. Conditions were chosen with no more than 20% of substrate conversion, and measurements were carried out in the linear range of product formation versus reaction time and enzyme concentration. The reactions were stopped by addition of a threefold excess of acetonitrile. After centrifugation in a microfuge for 5 min the samples were directly separated on RP-HPLC, consisting of a C18 stationary phase and an eluent of 30% acetonitrile in 0.1% ammonium acetate, pH 7.0. Substrate and product were monitored by UV absorbance at 240 nm and concentrations were calculated by referral to corresponding calibration curves.

Gel Permeation chromatography. A Superdex 200 HiLoad 26/60 column (Amersham Biosciences) equilibrated in 20 mM Tris-HCl, pH 8.0: 0.2 M NaCl: 1 mM TCEP was calibrated with high and low molecular weight gel filtration calibration kits (Amersham Biosciences). Ferritin, catalase, aldolase, albumin, ovalbumin, chymotrypsinogen and ribonuclease were dissolved in the same buffer as above and run through the gel filtration column in separate experiments according to the suppliers instructions. The same chromatographic procedure was then applied on purified 11β-HSD1 in a corresponding buffer supplemented with 0.05% Triton X-100. The apparent molecular weight of the protein-detergent complex was calculated by comparing its elution volume with those of the standard proteins.

Active site titration. Fractional velocities (dehydrogenase reaction, 50 μM cortisol, 100 μM NADP⁺) were measured in the presence of increasing amounts of the arylsulfonamidothiazole inhibitor BVT.24829 (Biovitrum). Data obtained were fitted by non-linear regression to Equation 1 (23)
vᵢ/vₒ =1−((E+I+Kᵢ^{app})−((E+I+Kᵢ^{app})²−4×E×I)^{1/2})×(2×E)⁻¹ (Eq. 1)
where vᵢ is the observed velocity in the presence of varied inhibitor concentrations, vₒ is the uninhibited velocity, E is the concentration of active enzyme, Kᵢ^{app} is the apparent inhibition constant, and I is the experimental inhibitor concentration.

Stability measurements. The conformational stability was examined by monitoring GuHCl-induced solvent inactivation of 11β-HSD1. Protein solutions (5-10 μM) were incubated overnight at room temperature in various concentrations of GuHCl (0-2 M) buffered with 50 mM Tris-HCl, pH 8.0. The inactivation of the enzyme was monitored by the decrease in dehydrogenase activity (25 μM cortisol, 50 μM NADP⁺) using the fluorimetric assay described above. The stability data were analyzed by nonlinear regression, fitting to the equation described by Santoro and Bolen (24) using the program GraFit, version 5.0 (Erithacus Software, Staines, UK). The midpoint concentration of inactivation, Cₘ, was determined from the ratio, ΔG^{H2O}/m, of the fitted parameters.

Analytical ultracentrifugation. Velocity sedimentation experiments were performed using an XL-A analytical ultracentrifuge (Beckman Instruments, Palo Alto, Calif.) with an An60-Ti rotor. 11β-HSD1 in 50 mM Tris-HCl, pH 8.0: 1 mM TCEP and increasing concentrations of GuHCl (0-2.0 M) was centrifuged at 40,000 rpm. Solutions containing the same components except the protein were used as references. Scans were collected every 5 min at 280 nm absorption wavelength and the radial distance of the sedimenting boundary r was determined. The sedimentation coefficient was calculated by the gradient of the plot of ln(r) vs ω²t, where ω was the angular velocity and t was the time.

Dynamic Light Scattering. 11β-HSD1, purified by His-bind affinity chromatography in the absence of detergents, was incubated for approximately 1 h at room temperature with various detergents in 50 mM Tris-HCl, pH 8.0: 1 mM TCEP. Subsequently, solutions were analyzed at 22° C. using a DynaPro-801 light scattering/molecular sizing instrument (Protein Solutions, Charlottesville, Va.). The measurements were taken at both the critical micelle concentration (CMC) and 0.2×CMC. The degree of polydispersion was calculated using either the mono-modal or the bi-modal assumption within the instrumentation software.

Inhibitors (antagonists) of 11β-HSD1. The inhibitor designated BVT.24829 (3-chloro-2-methyl-N-{5-methyl-4-[2-(3-oxo-4-morpholinyl)ethyl]-1,3-thiazol-2-yl}benzenesulfonamide) was prepared as generally described in WO 01/90090. The inhibitor designated BVT.3498 (3-chloro-2-methyl-N-{4-[2-(3-oxo-4-morpholinyl)ethyl]-1,3-thiazol-2-yl}benzenesulfonamide) was prepared as described in WO 01/90090 (cf. Example 210A). The inhibitor designated BVT.4584 (N,N-diethyl-(3-{[(4-propylphenyl)sulfonyl]amino}thien-2-yl)carboxamide) was prepared as generally described in WO 03/044009. The inhibitor designated BVT.2733 (3-Chloro-2-methyl-N-{4-[2-(4-methyl-1-piperazinyl)-2-oxoethyl]-1,3-thiazol-2-yl}benzenesulfonamide) was prepared as described in WO 01/90090 (cf. Example 172A) and in Ref. No. 35.

BVT.24829 and BVT.3498 have Kᵢ values for 11β-HSD1 in the nanomolar range, while BVT.4584 and BVT.2733 have Kᵢ values in the micromolar range.

Surfactant. The non-ionic surfactant Triton® X-100 (CAS # [9002-93-1]; chemical name: p-(1,1,3,3-Tetramethylbutyl)phenol ethoxylate) was purchased from Anatrace, Maumee, Ohio.

### Example 1

### High-Level Production of Soluble Recombinant 11β-HSD1

Construction of pET28a (Novagen, Madison, Wis.) derivatives for expression of the catalytic domain of human, rat and guinea pig 11β-HSD1 in E. coli has been described previously (21, 22). For all orthologues, the first 23 amino acid residues (19 for the rat variant) of the protein, which includes the distal N-terminus and the transmembrane domain, were omitted and the remaining sequence was placed in frame behind a His₆ tag. The verified constructs were used to transform the E. coli expression strain BL21(DE3). The amino acid sequences of the encoded human (SEQ ID NO:1); rat (SEQ ID NO:2) and guinea pig (SEQ ID NO:3) 11β-HSD1 variants are shown in the Sequence Listing.

Two different strategies were employed to optimize production of soluble 11β-HSD1. For expression of the human and rat variant, the cells were grown in shake flasks using Terrific Broth medium supplemented with 50 μg/ml kanamycin and 34 μg/ml chloramphenicol. To increase the yield of soluble human and rat 11β-HSD1, the BL21(DE3) cells were co-transformed with the plasmid pBV530, harboring the genes for the E. coli chaperonin GroEL/ES under control of the araBAD promoter. The cells were then grown at 25° C. to a cell density corresponding to an A₆₀₀ value of 0.5-1.0. To induce expression of GroEL/ES, arabinose was added to a final concentration of 0.05%. 45-60 minutes after induction of the chaperonin, 0.2 mM isopropyl-β-D-thiogalactoside (IPTG) was added to initiate production of 11β-HSD1. To obtain optimal production of soluble enzyme, 0.5-50 μM of an arylsulfonamidothiazole derivative known to selectively inhibit 11β-HSD1 was included to the growth medium. Incubation was continued at 18° C. for 12-16 h before the cells were harvested by low speed centrifugation.

For expression of the guinea pig variant, the cells were grown in a 7-liter fermentor (Belach Bioteknik, Solna, Sweden) containing 4,5 liters of minimal medium (M9) supplemented with 5 g/l yeast extract and 5 g/l glucose The temperature was set at 25° C., the dissolved oxygen tension at 30% and pH controlled at 7.0. Protein production was initiated by addition of 0.1 mM IPTG at an A₆₀₀ of 20. The temperature was lowered to 20° C., and the cells were cultivated for additional 20 h. After fermentation, the cells were pelleted by centrifugation at 8,000 g for 20 min at 4° C. For all 11β-HSD1 orthologues, cells were lysed by sonication just prior to purification, and cellular debris was removed by centrifugation.

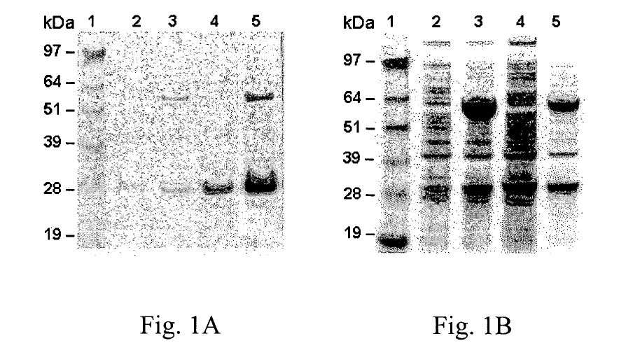

Although a large amount of protein was produced, only a fraction of soluble 11β-HSD1 was found in the cleared lysate (FIGS. 1A and 1B, lane 2). In accordance with previous observations (21,36), it was found that lowering the growth temperature during induction increased the level of production of soluble protein. For human and rat 11β-HSD1, co-overexpression of the E. coli GroEL/ES chaperonin resulted in more soluble enzyme (FIGS. 1A and 1B, lane 3). In addition, for both variants the yield of soluble protein was further increased by addition of the selective arylsulfonoamidothiazole inhibitor BVT.24829 (3-chloro-2-methyl-N-{4-[2-(3-oxo-4-morpholinyl)ethyl]-1,3-thiazol-2-yl}benzenesulfonamide) at the time of induction (FIGS. 1A and 1B, lane 4). As estimated by SDS-PAGE analysis, the yield of soluble human 11β-HSD1 was improved approximately 60-fold by GroEL/ES co-overexpression in combination with addition of BVT.24829 to the growth medium (FIG. 1A, lane 5). Production of soluble guinea pig enzyme was only improved approximately 2-fold by co-overexpression of GroEL/ES (data not shown). Moreover, addition of the inhibitor BVT.4584 (N,N-diethyl-(3- {[(4-propylphenyl)sulfonyl]amino}thien-2-yl)carboxamide) (having a Kᵢ value in the micromolar range) to the growth medium did not significantly enhance the yield of soluble guinea pig enzyme. Therefore, a fermentation strategy was used for obtaining optimal heterologous expression of guinea pig 11β-HSD1.

### Example 2

### Purification of Recombinant 11β-HSD1

Immobilized metal affinity chromatography (IMAC) has previously been used to purify both the full-length enzyme and the truncated variant lacking the transmembrane domain (15, 21, 22). As described in Example 1, recombinant 11β-HSD1 variants were designed to contain a hexa-histidine affinity tag, allowing affinity purification without disturbing the enzymatic activity.

The cleared lysate, obtained according to Example 1, was applied directly to a HiTrap Chelating HP column using an ÄKTA protein purifier system (Amersham Biosciences, Uppsala, Sweden). The column was equilibrated with 50 mM Tris-HCl, pH 8.0: 2 mM TCEP (Tris(2-carboxyethyl)phosphine): 5 mM imidazole: 5% (w/v) glycerol, 300 mM NaCl, and then washed with the same buffer containing 50 and 85 mM imidazole, respectively, until the absorbance at 280 nm returned to the baseline. At this point, the target protein was not eluted, but the column was instead re-equilibrated with the same buffer as above.

For purification of the human 11β-HSD1, the bound enzyme was incubated overnight with column buffer containing 2 mM ATP, 0.5 mM MgCl₂ and 0.05% Triton X-100. In addition, to stabilize the protein during the incubation and in subsequent purification steps, a low concentration (0.5-25 μM) of the arylsulfonoamidothiazole inhibitor BVT.24829 was included in the buffer. The next day the protein was eluted from the column with a stepwise gradient of imidazole. Fractions containing 11β-HSD1 were pooled, concentrated and applied on a prepacked Superdex 200 HiLoad 26/60 column (Amersham Biosciences) equilibrated with 20 mM Tris-HCl, pH 8.0: 2 mM TCEP: 5% (w/v) glycerol: 0.05% Triton X-100 and BVT.24829. Fractions containing pure 11β-HSD1 were pooled and concentrated using a 30000 MW cutoff Amicon Ultra concentration device (Millipore, Bedford, Mass.).

For purification of the guinea pig orthologue, the same procedure was used with the following exception: Enzyme bound to the HiTrap Chelating column was incubated in the presence of 0.5 M GuHCl (guanidine hydrochloride), 20 μM NADP⁺ and 10 μM of the arylsulfonoamidothiazole inhibitor BVT.4584, but in the absence of ATP and MgCl₂. Also in this case, the additions were included throughout the purification scheme.

Both enzyme variants were 95-99% homogeneous as determined by SDS-polyacrylamide gel electrophoresis. Protein concentrations were estimated with the Bio-Rad protein assay, standardized with bovine serum albumin, complemented with amino acid analysis on a AminoQuant II system (Hewlett Packard, Wilmington, Del.) after hydrolysis of samples in 6M HCl: 0.1% phenol.

In the absence of detergent, 11β-HSD1 eluted from the IMAC column together with a large amount of E. coli proteins. A single peak emerged with the void volume in the subsequent gel permeation chromatography step, indicating that 11β-HSD1 existed in large soluble aggregates associated with the contaminating E. coli proteins. Also dynamic light scattering measurements indicated that most of the protein was in the form of high molecular weight oligomers in the absence of detergent, a characteristic of many intrinsic membrane proteins. Active site titration was carried out with a selective and potent inhibitor of human 11β-HSD1 yielding a fraction of approximately 20% active enzyme molecules of the total protein content.

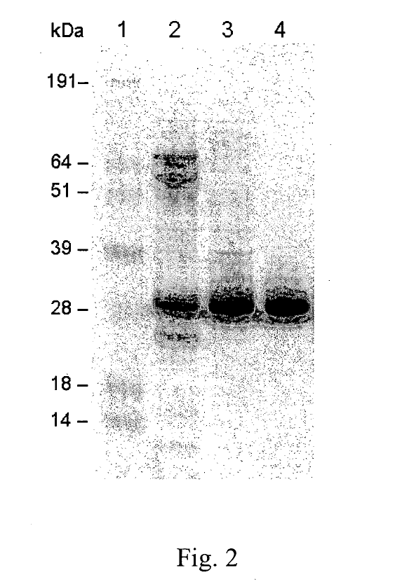

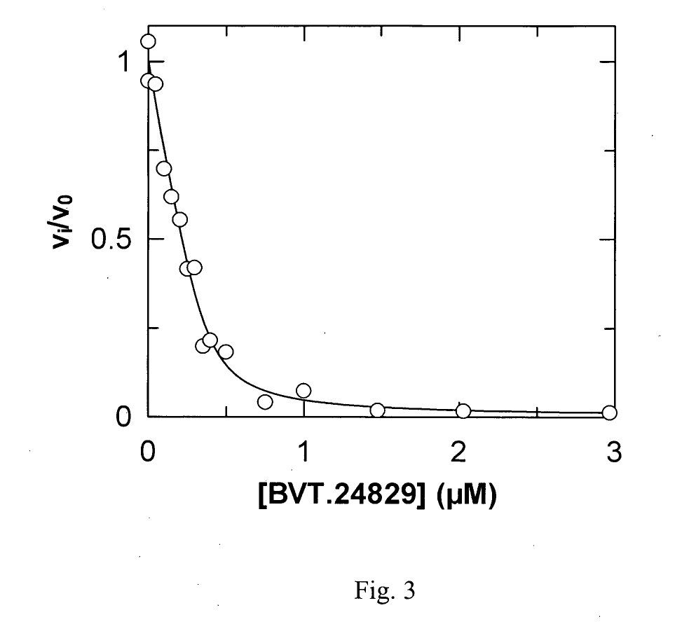

Various detergents were screened for their ability to solubilize the material obtained from the IMAC purification, including Triton X-100, CHAPS, C₁₂E₈, octylglucoside (OG) and dodecylmaltoside (DDM). For human 11β-HSD1, Triton X-100 was found to be most effective at solubilizing the aggregates. Gel permeation chromatography in buffer containing 0.05% Triton X-100 efficiently separated unsolubilized material from 11β-HSD1. The apparent molecular weight of the 11β-HSD1—Triton X-100 complex was estimated to 120 kDa by comparing its elution volume with those of the standard proteins. The 11β-HSD1 was more than 95% pure based on Coomassie-stained SDS-PAGE (FIG. 2, lane 4). Only peak fractions with A₂₈₀ above 0.4 were collected and used for further experiments, since they contained the purest, most concentrated protein. Active site titration of the main peak fraction yielded a conservative value of around 75% of active enzyme molecules (FIG. 3), an almost 4-fold improvement in specific activity compared with the material prior to solubilization.

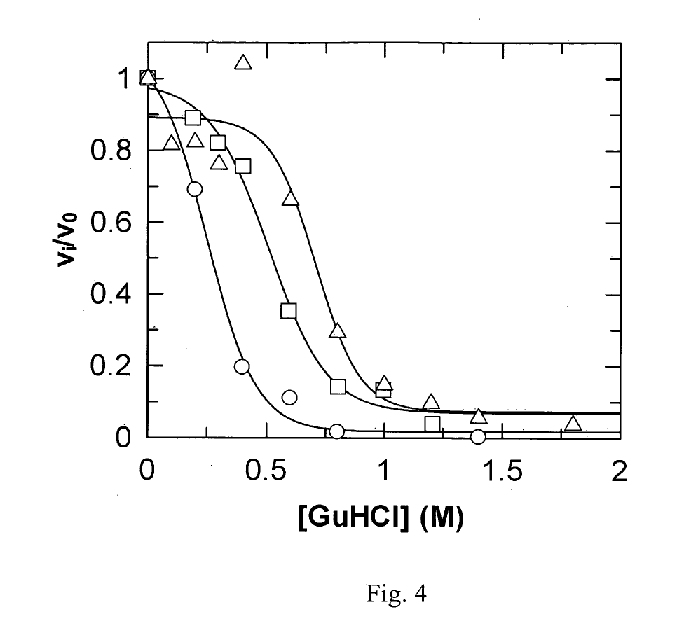

In contrast to the human enzyme, guinea pig 11β-HSD1 was only partially extracted by Triton X-100 from the soluble aggregates. This prompted us to analyze the possibility of enhancing the stability of the guinea pig variant to enable further purification under more harsh conditions. It was found that addition of BVT.4584 and the cofactor NADP⁺ incrementally increased the unfolding midpoint of guinea pig 11β-HSD1 from 0.5 to 1.0 M GuHCl (Table I). Also the stability of the human enzyme could be increased in a stepwise manner. Here, addition of BVT.24829 and NADP⁺ improved the midpoint of the inactivation profile from 0.2 to 0.7 M GuHCl (FIG. 4). Gel permeation chromatography of the IMAC-purified guinea pig enzyme in buffer containing 0.5 M GuHCl, 0.05% Triton X-100, 25 μM BVT.4584, 50 μM NADP⁺ resulted in a similar purity (>95%) as the human variant (not shown). The protein was stable for several weeks when stored at 4° C.

TABLE I
Effects of addition of inhibitor and cofactor on the midpoint
concentration, Cₘ, for GuHCl-induced inactivation of
human and guinea pig 11β-HSD1 as monitored by cortisol
dehydrogenation activity measurements.

| | |
|---|---|
| Human | Guinea pig |

Cₘ(M)

| | | |
|---|---|---|
| Control (no addition) | 0.25 | 0.51 |
| Inhibitor^{a} | 0.51 | 0.75 |
| Inhibitor + Cofactor^{b} | 0.71 | 0.98 |

^{a}One equivalent of the high affinity inhibitor BVT.24829 was added to human variant, while 25 μM of BVT.4584 was added to the guinea pig variant.
^{b}50 μM NADP⁺.

### Example 3

### Oligomerization State of Purified Recombinant 11β-HSD1

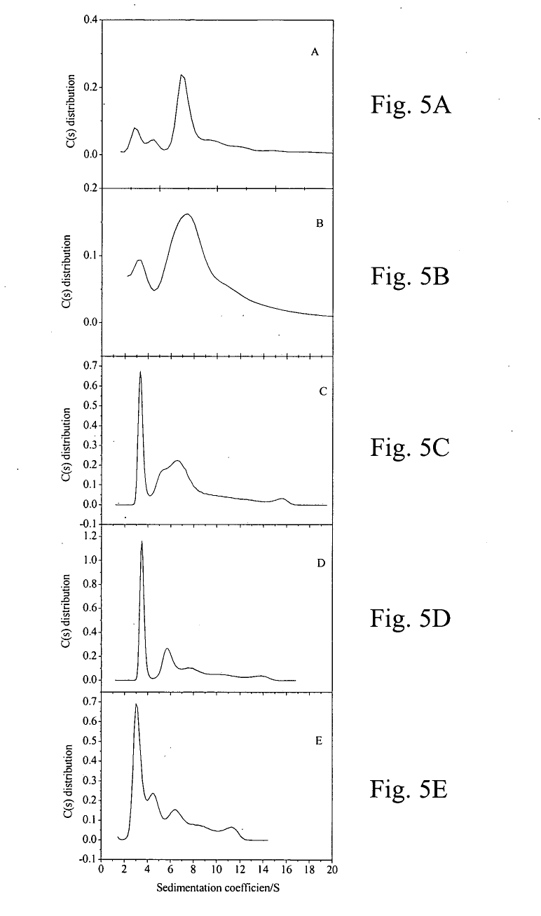

The association state of 11β-HSD1 was investigated by analytical ultracentrifugation. Sedimentation velocity runs of 11β-HSD1 preparations prior to solubilization revealed several oligomeric populations (FIG. 5A), indicating a polydisperse solution in agreement with previous conclusions. When incubated in buffer containing 0.4 M GuHCl, the guinea pig enzyme appeared to be constituted by two major populations (FIG. 5B). This behavior was even more pronounced at 0.6 M GuHCl (FIG. 5C). A molecular weight of approximately 60 kDa can be derived from the sedimentation coefficient of the larger peak, corresponding to a dimeric 11β-HSD1 population, while the second peak may originate from a smaller portion of tetrameric enzyme.

Importantly, 11β-HSD1 purified in the presence of Triton X-100 and/or GuHCl retains enzymatic activity. For human 11β-HSD1, it was found that the enzyme exhibits high specific activity, displaying at least 75% functional active sites. Although the gel permeation chromatography yielded an apparent molecular weight of approximately 120 kDa, it is unlikely that it would originate from a tetramer of 11β-HSD1. The apparent molecular weights of proteins estimated in presence of detergents are usually significantly higher than the true values due to formation of detergent-protein complexes (34). Therefore, the calculated molecular weight of around 60 kDa, derived from analytical ultracentrifugation in absence of detergent, should be much closer to the true value of this enzyme and indicates that 11β-HSD1 is active as a dimer in vivo.

### REFERENCES

1. Stewart, P. M. and Krozowski, Z. S. (1999) Vitam Horm 57, 249-324
2. Labrie, F. (1991) Mol Cell Endocrinol 178, C113-118.
3. Labrie, F., Luu-The, V., Lin, S. X., Simard, J. and Labrie, C. (2000) Trends Endocrinol Metab 11, 421-427.
4. Nobel, S., Abrahmsén, L. and Oppermann, U. Eur J Biochem 268, 4113-4125.
5. Seckl, J R. and Walker, B. R. (2001) Endocrinology 142, 1371-1376.
6. Rabbitt, E. H., Lavery, G. G., Walker, E. A., Cooper, M. S., Stewart, P. M. and Hewison, M. (2002) Faseb J 16, 36-44.
7. Oppermann, U. C., Persson, B. and Jörnvall, H. (1997) Eur J Biochem 249, 355-360.
8. Alberts P., Nilsson, C., Selen, G., Engblom, L. O., Edling, N. H., Norling, S., Klingstrom, G., Larsson, C., Forsgren, M., Ashkzari, M., Nilsson, C. E., Fiedler, M., Bergqvist, E., Ohman, B., Björkstrand, E. and Abrahmsén, L. B. (2003) Endocrinology 144, 4755-4762.
9. Stulnig, T. M. and Waldhausl, W. Diabetologia In press.
10. Oppermann, U., Filling, C., Hult, M., Shafqat, N., Wu, X., Lindh, M., Shafqat, J., Nordling, E., Kallberg, Y., Persson, B. and Jörnvall, H. (2003) Chem Biol Interact 143-144, 247-253.
11. Ghosh D., Pletnev, V. Z., Zhu, D. W., Wawrzak, Z., Duax, W. L., Pangborn, W., Labrie, F. and Lin, S. X. (1995) Structure 3, 503-513.
12. Ghosh, D., Wawrzak, Z., Weeks, C. M., Duax, W. L. and Erman M. (1994) Structure 2, 629-640.
13. Tanaka, N., Nonaka, T., Tanabe, T., Yoshimoto, T., Tsuru, D. and Mitsui, Y. (1996) Biochemistry 35, 7715-7730.
14. Varughese, K. I., Xuong, N. H., Kiefer, P. M., Matthews, D. A. and Whiteley, J. M. (1994) Proc Natl Acad Sci USA 91, 5582-5586.
15. Nobel, C. S., Dunas, F., and Abrahmsén, L. B. (2002) Protein Expr Purif 26, 349-356.
16. Hult M., Nobel, C. S., Abrahmsén, L., Nicoll-Griffith, D. A., Jörnvall, H. and Oppermann, U. C. (2001) Chem Biol Interact 130-132, 805-814.
17. Jamieson, P. M., Chapman, K. E., Edwards, C. R. and Seckl, J. R. (1995) Endocrinology 136, 4754-4761.
18. Bujalska, I. J., Walker, E. A., Tomlinson, J. W., Hewison, M. and Stewart, P. M. (2002) Endocr Res 28, 449-461.
19. Lakshmi V. and Monder C. (1988) Endocrinology 123, 2390-2398.
20. Agarwal, A. K., Tusie-Luna, M. T., Monder, C. and White, P. C. Mol Endocrinol 4, 1827-1832.
21. Walker, E. A., Clark, A. M., Hewison, M., Ride, J. P. and Stewart, P. M. (2001) J Biol Chem 276, 21343-21350.
22. Shafqat, N., Elleby, B., Svensson, S., Shafqat, J., Jörnvall, H., Abrahmsén, L. and Oppermann, U. (2003) J Biol Chem 278, 2030-2035.
23. Morrison, J. F. (1969) Biochim Biophys Acta 185, 269-280.
24. Santoro, M. M. and Bolen, D. W. (1988) Biochemistry 27, 8063-8068.
25. Blum, A., Raum, A., Martin, H. and Maser, E. (2001) Chem Biol Interact 130-132, 749-759.
26. Blum, A., Martin, H. J. and Maser, E. (2000) Toxicology 144, 113-120.
27. Luo, Z. and Hua, Z. C. (1998) Biochem Mol Biol Int 46, 471-477.
28. Lamark, T, Ingebrigtsen, M., Bjornstad, C., Melkko, T., Mollnes, T. E. and Nielsen, E. W. (2001) Protein Expr Purif 22, 349-358.
29. Lee, K. H., Kim, H. S., Jeong, H. S. and Lee, Y. S. (2002) Biochem Biophys Res Commun 298, 216-224.
30. Todd, M. J. and Freire, E. (1999) Proteins 36, 147-156.
31. Lemieux, M. J., Reithmeier, R. A. and Wang, D. N. (2002) J Struct Biol 137, 322-332.
32. Maser, E. and Bannenberg, G. (1994) J Steroid Biochem Mol Biol 48, 257-263.
33. Oppermann, U. C., Mobus, E., Nagel, G. and Maser, E. (2000) Toxicology 144, 63-69.
34. Lakshmi, V. and Monder, C. (1985) J Steroid Biochem 22, 331-340.
35. Barf, T. et al. (2002) J Med Chem 45, 3813-3815.
36. Georgiou, G. and Valax, P. (1996) Curr Opin Biotechnol 7, 190-197.
37. Hartl, F. U. and Hayer-Hartl, M. (2002) Science 295, 1852-1858.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

1. A method for preparing a soluble polypeptide, the method comprising:
providing a host cell comprising a nucleic acid sequence encoding a recombinant soluble polypeptide comprising at least one ligand binding site;
culturing the host cell under conditions whereby the polypeptide is produced, wherein the cell culture medium comprises a non-proteinaceous ligand that binds to the at least one ligand binding site of the polypeptide; and
recovering the polypeptide from the cell culture medium.

2. The method of claim 1, wherein the polypeptide is a recombinant soluble human polypeptide.

3. The method of claim 1, wherein the non-proteinaceous ligand binds to the polypeptide with a Kᵢ value below 100 μM.

4. The method of claim 1, wherein the non-proteinaceous ligand is an inhibitor of an enzymatic activity of the polypeptide.

5. The method of claim 1, wherein the host cell comprises an agent that assists protein folding.

6. The method of claim 5, wherein the host cell comprises a recombinant vector encoding the agent that assists protein folding.

7. The method of claim 5, wherein the agent that assists protein folding is a chaperonin.

8. The method of claim 1, wherein the host cell is an E. coli cell.

9. The method of claim 7, wherein the chaperonin is GroEL/ES.

10. The method of claim 1, wherein the polypeptide is a recombinant soluble human 11β-hydroxysteroid dehydrogenase type 1 (11β-HSD1) polypeptide comprising amino acids 22 to 290 of SEQ ID NO:1 or a fragment thereof exhibiting oxidoreductase activity.

11. The method of claim 1, wherein the polypeptide consists essentially of amino acids 22 to 290 of SEQ ID NO:1 and optionally a tag for purification of the polypeptide.

12. The method of claim 10, wherein the non-proteinaceous ligand is an inhibitor of an enzymatic activity of 11β-HSD1.

13. The method of claim 10, wherein the non-proteinaceous ligand is an inhibitor of the oxidoreductase activity of 11β-HSD1.

14. The method of claim 10, wherein the non-proteinaceous ligand is an inhibitor of the dehydrogenase activity of 11β-HSD1.

15. The method of claim 10, wherein the non-proteinaceous ligand is an arylsulfonoamidothiazole derivative.

16. The method of claim 1, further comprising subjecting the recovered polypeptide to at least one chromatography step in the presence of (i) a non-proteinaceous ligand that binds to the at least one ligand binding site of the polypeptide; and (ii) a solubilizing agent.

17. The method of claim 16, wherein the solubilizing agent is p-(1,1,3,3-tetramethylbutyl)phenol ethoxylate.

18. A method for obtaining crystals of 11β-HSD1, the method comprising:
(i) providing a monodisperse preparation of a recombinant soluble human 11β-HSD1 polypeptide, obtained by the method of claim 17; and
(ii) crystallizing the polypeptide.
